(19) United States
(12) Patent Application Publication
(10) Pub. No.: US 2022/0110869 A1
(43) Pub. Date: Apr. 14, 2022

(54) TOPICAL MINOCYCLINE FOAMABLE COMPOSITIONS

(71) Applicant: Sol-Gel Technologies Ltd., Ness Ziona (IL)

(72) Inventors: Karine NEIMANN, Ness Ziona (IL); Danil FINKEL-MOISEEV, Rehovot (IL); Moshe ARKIN, Kfar Shmaryahu (IL); Adina HAIMOV, Bnei-Ayish (IL)

(73) Assignee: Sol-Gel Technologies Ltd., Ness Ziona (IL)

(21) Appl. No.: 17/289,236

(22) PCT Filed: Oct. 28, 2019
(86) PCT No.: PCT/IL2019/051164

§ 371 Date: Apr. 28, 2021

Related U.S. Application Data
(60) Provisional application No. 62/795,010, filed on Jan. 21, 2019.
(60) Provisional application No. 62/792,445, filed on Jan. 15, 2019.
(60) Provisional application No. 62/752,267, filed on Oct. 29, 2018.

(51) Int. Cl. A61K 9/12; A61K 31/65; A61K 47/44; A61K 47/12

(52) U.S. Cl. CPC A61K 9/122; A61K 47/12; A61K 47/44; A61K 31/65

(57) ABSTRACT

Disclosed are simple yet stable and effective compositions comprising from about 1% to about 5% by weight of the composition of a minocycline or mixtures thereof and pharmaceutically acceptable ingredients comprising from about 60% to about 99% by weight of the composition of at least one hydrophobic oil, and from about 5% to about 25% by weight of the composition of at least one fatty alcohol, wherein the at least one hydrophobic oil and the at least one fatty alcohol are sufficient to ensure the pharmaceutically acceptable assay stability of the minocycline in the composition under 3 months accelerated stability conditions at 40° C./75% RH. Also disclosed are methods of treatment of acne, rosacea and impetigo.

### FIELD OF THE INVENTION

The present invention is in the field of pharmaceutical compositions and discloses a stable topical foamable composition comprising at least one pharmaceutical active agent, wherein the composition is useful for treating a skin disorder. More specifically, the present invention discloses stable minocycline foamable compositions for use in skin disorders selected from acne, rosacea and impetigo.

### BACKGROUND OF THE INVENTION

Topical pharmaceutical compositions formulated as foams or foamable compositions and comprising various active agents are known in the art and recently have gained in popularity (J Pharm Pharmacol. 2010 June; 62(6):678-84).

One of the active agents in foamable compositions is minocycline, a broad-spectrum tetracycline antibiotic, in commercial use since 1971.

Minocycline is unstable in the presence of water. A tetracycline stability study (Honnorat-Benabbou et al, J Mater Sci Mater Med. 2001 February; 12(2):107-10) included an assay showing that minocycline hydrochloride amounts fell by about 10% in water in three days, which points to minocycline instability in water.

Minocycline-containing topical foamable compositions have been disclosed in several patents, such as U.S. Pat. Nos. 8,343,945, 8,871,184, 8,865,139, 8,992,896, 8,618,081 and 8,945,516, 9,675,700, 9,849,142 and 10,029,013 (to Foamix Pharmaceuticals Ltd.). The surfactant-free compositions of the above patents comprise, in addition to a list of active agents (including a minocycline), a large number of ingredients, belonging to several ingredient types such as waxes, fatty acids, shea butter, short chain alcohols, polyols, polar solvents, polymers, hydrocarbon-based oils, mineral oils and petrolatum.

Some of the above ingredients and ingredient types are irritants or are deleterious to the active agent or the skin.

A further object of the present invention is the formulation of minocycline using only fatty alcohols as foaming agents. Such formulations are not known in the art and their addition to the formulary would be advantageous as there remains an unmet need for simple, yet stable, foamable and effective topical foamable compositions devoid of ingredients which are irritants or are deleterious to the active agent or the skin in a topical formulation.

### SUMMARY OF THE INVENTION

Quite surprisingly, the present inventors have discovered that certain compositions comprising in addition to the at least one active agent and hydrophobic oils, only one type of ingredient—fatty alcohols—should be foamable, stable and effective. Fatty alcohols and hydrophobic oils are mild, not irritant or deleterious to the skin or the active ingredient yet the compositions are stable, foamable and effective. That fatty alcohols alone, in absence of waxes, fatty acids, shea butter and polymers lead to stable, foamable and effective compositions is novel and surprising. The present invention provides a stable foamable composition comprising from about 1% to about 5% by weight of the composition of at least one active agent. Also included are pharmaceutically acceptable ingredients comprising from about 65% to about 99% by weight of the composition. The pharmaceutically acceptable ingredients include from about 60% to about 90% of at least one hydrophobic oil. The formulation further includes from about 5% to about 25% by weight of at least one fatty alcohol. The at least one hydrophobic oil and the at least one fatty alcohol comprise in total from about 65% to about 99% by weight of the composition.

The present invention provides a stable minocycline composition comprising: from about 1% to about 5% by weight of minocycline;

from about 60% to about 90% by weight of the composition of at least one hydrophobic oil, and
from about 5% to about 25% by weight of the composition of at least one fatty alcohol;
wherein the at least one hydrophobic oil and the at least one fatty alcohol comprise in total from about 65% to about 99% by weight of the composition;
wherein the at least one hydrophobic oil and the at least one fatty alcohol are in a weight ratio of from about 4:1 to about 8:1; and
wherein the composition is essentially free of water, waxes, fatty acids, shea butter, short chain alcohols, polyols, polar solvents, polymers, hydrocarbon-based oils, mineral oils and petrolatum.

In another embodiment, the stable minocycline composition is a foamable composition, and the composition further comprises at least one propellant, wherein the ratio of the minocycline composition to propellant is from about 100:3 to about 100:30, preferably 100:10, and wherein upon dispensing, the foamable composition forms a breakable foam that breaks easily upon application of shear force. In another embodiment, the foamable composition is stable and the minocycline does not decompose more than 3% for at least about 3 months under accelerated stability conditions at 40° C./75% RH.

The at least one hydrophobic oil and the at least one fatty alcohol are present in sufficient amounts and in a ratio sufficient to ensure the pharmaceutically acceptable assay stability of the minocycline in the composition for at least about 3 months under accelerated stability conditions of 40° C./75% RH.

In some embodiments the at least one active agent is a minocycline selected from minocycline, minocycline hydrochloride, a hydrate, a solvate and a mixture thereof.

The composition of this invention comprises a small number of pharmaceutically acceptable ingredients, comprising in addition to hydrophobic oils only one type of ingredient, fatty alcohols, which are not irritants or deleterious to the skin or to the active ingredient, yet together they yield a composition which is foamable, stable and effective.

As the compositions are essentially free of water, the minocycline-containing compositions of this invention are stable, e.g., the minocycline in these formulations is not degraded as rapidly as it is in prior reported formulations.

The stable minocycline composition may further comprise from about 0.1% to about 0.5% by weight of the composition of silicone oxide, wherein the silicon oxide is, for example, Syloid™ or Aerosil™.

A propellant may be added to the minocycline composition of this invention, wherein the ratio of composition to propellant is from about 100:3 to about 100:30, preferably about 100:10, and wherein upon dispensing, the foamable composition forms a breakable foam that breaks easily upon application of shear force. Exemplary propellants include, without limitation, hydrocarbons, e.g., n-butane, isobutane, propane, n-pentane and mixtures thereof.

In addition to the above ingredients, the minocycline composition of this invention may further comprise from about 0.1% to about 20% by weight of the composition of at least one fatty acid selected from stearic acid, palmitic acid and mixtures thereof, in which case it may also contain a polymer. Exemplary polymers are selected from the group consisting of a polypropylene glycol, polyethylene glycol, ethylcellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

An exemplary polymer to be used in the composition is polyethylene glycol, e.g. PEG 3350.

The composition of this invention is useful for treatment of a dermatological, topical disorder, e.g., acne, rosacea and impetigo.

### DETAILED DESCRIPTION OF THE PRESENT INVENTION

The present invention provides a stable foamable composition comprising from about 1% to about 5% by weight of the composition of at least one active agent. Also included are pharmaceutically acceptable ingredients comprising from about 60% to about 99% by weight of the composition. The pharmaceutically acceptable ingredients include from about 60% to about 90% of at least one hydrophobic oil. The formulation further includes from about 5% to about 25% by weight of at least one fatty alcohol. The at least one hydrophobic oil and the at least one fatty alcohol comprise in total from about 65% to from about 99% by weight of the composition.

The present invention provides a stable minocycline composition comprising: from about 1% to about 5% by weight of minocycline;

from about 60% to about 90% by weight of the composition of at least one hydrophobic oil, and
from about 5% to about 25% by weight of the composition of at least one fatty alcohol;
wherein the at least one hydrophobic oil and the at least one fatty alcohol comprise in total from about 65% to about 99% by weight of the composition;
wherein the at least one hydrophobic oil and the at least one fatty alcohol are in a weight ratio of from about 4:1 to about 8:1; and
wherein the composition is essentially free of water, waxes, fatty acids, shea butter, short chain alcohols, polyols, polar solvents, polymers, hydrocarbon-based oils, mineral oils and petrolatum.

In another embodiment, the stable minocycline composition is a foamable composition, and the composition further comprises at least one propellant, wherein the ratio of the minocycline composition to propellant is from about 100:3 to about 100:30, preferably 100:10, and wherein upon dispensing, the foamable composition forms a breakable foam that breaks easily upon application of shear force. In another embodiment, the foamable composition is stable and the minocycline does not decompose more than 3% for at least about 3 months under accelerated stability conditions at 40° C./75% RH.

The at least one hydrophobic oil and the at least one fatty alcohol are present in sufficient amounts and in a ratio sufficient to ensure the stability of the minocycline in the composition for at least about 3 months under accelerated stability conditions of 40° C./75% RH, and at least about 2 years stability at room temperature.

In some embodiments, the compositions of this invention, upon packaging, are essentially free of water, waxes, fatty acids, shea butter, short chain alcohols, polyols, polar solvents, polymers, hydrocarbon-based oils, mineral oils and petrolatum.

In some embodiments the at least one active agent is a minocycline selected from minocycline, minocycline hydrochloride, a hydrate, a solvate and a mixture—thereof.

In some other embodiments, the at least one active agent for the composition of this invention is selected from adapalene, adipic acid, an acaricide, an active herbal extract, an age spot and keratose removing agent, an alpha hydroxy acid, an analgesic agent, an androgen, an anesthetic, an anti-wrinkle agent, an antiacne agent, an antiaging agent, an antiallergic agent, an antiandrogen agent, an antiapoptotic agent, an antibacterial agent, an antibiotic, an anti-burn agent, an anticancer agent, an anti-dandruff agent, an antidepressant, an anti-dermatitis agent, an anti-edemic anent, an antifungal agent, an antihelminth agent, an antihistamine, an anti-hyperkeratosis agent, an anti-infective agent, an anti-inflammatory agent, an anti-irritant, an antilipemic agent, an antimicrobial agent, an antimycotic agent, an antioxidant, an antiparasitic agent, an anti-photoaging agent, an anti-photodamaging agent, an antiproliferative agent, an antipruritic agent, an anti-psoriatic agent, an anti-rosacea agent, an anti-seborrheic agent, an antiseptic agent, an anti-swelling agent, an antiviral agent, an anti-wart agent, an anti-wrinkle agent, an anti-yeast agent, azelaic acid, benzoyl peroxide, a beta-hydroxy acid, calcitriol, a cardiovascular agent, a chemotherapeutic agent, a corticosteroid, a dicarboxylic acid, a dihydrotestosterone inhibitor, a disinfectant, doxycycline, an estrogen, a fungicide, fumaric acid, glycolic acid, a hair growth regulator, a haptene, a herbal extract (comprising an active substance), a hormone, a hydroxy acid, an immunogenic substance, an immunomodulator, an immuno-regulating agent, an immunostimulant, an immunosuppressant, an immuno-suppressive agent, an insect repellent, an insecticide, iron oxide, ivermectin, a keratolytic agent, lactic acid, a lactam, lidocaine, a local anesthetic agent, a minocycline, a mitocide, mometasone furoate, a neuropeptide, a non-steroidal anti-inflammatory agent, an organo-metallic compound, an oxidizing agent, and organo-boron compound, a pediculicide, a peptide, a pesticide, a photodynamic therapy agent, a progesterone, a prostaglandin, a protein, a radical scavenger, a retinoid, a sedative agent, a scabicide, sebacic acid, a sedative, a sedative agent, a self-tanning agent, silver, a silver compound, a skin protective agent, a skin whitening agent, a steroid, a steroidal anti-inflammatory agent, tretinoin, tazarotene, a testosterone inhibitor, a tetracycline antibiotic, a vasoactive agent, a vasoconstrictor, a vasodilator and mixtures thereof.

The composition of this invention comprises a small number of pharmaceutically acceptable ingredients, comprising in addition to the hydrophobic oils only one type of ingredient, fatty alcohols, which are not irritants or deleterious to the skin or to the active ingredient in the formulation, yet provide a composition which is stable, foamable and effective.

The compositions of this invention exhibit excellent stability in an accelerated stability test at 40° C./75% RH.

As the compositions are essentially free of water, the minocycline-containing compositions of this invention are stable.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

The composition may further comprise from about 0.1% to about 0.5% by weight of the composition of silicone oxide. A typical silicon oxide is Syloid™ or Aerosil™.

A propellant may be added to the composition of this invention, wherein the ratio of composition to propellant is from about 100:3 to about 100:30, preferably 100:10, and wherein upon dispensing, the foamable composition forms a breakable foam that breaks easily upon application of shear force. An exemplary propellant is selected from hydrocarbons, e.g., n-butane, isobutane, propane, n-pentane and mixtures thereof.

In addition to the above ingredients, the composition of this invention may further comprise from about 0.1% to about 20% by weight of the composition of at least one fatty acid selected from stearic acid, palmitic acid and mixtures thereof, in which case it will contain also a polymer, such as a polyethylene glycol.

The composition comprising a minocycline of this invention is useful for treatment of a topical disorder, selected from acne, rosacea and impetigo.

Minocycline is unstable in the presence of water. A tetracyclines stability study (Honnorat-Benabbou et al, J Mater Sci Mater Med. 2001 February; 12(2):107-10) has shown that minocycline hydrochloride lost up to about 10% of the total minocycline in water in three days.

Minocycline-containing topical foamable compositions have been disclosed in several patents, such as U.S. Pat. Nos. 8,871,184, 8,865,139, 8,992,896, 8,618,081 and 8,945,516, 9,675,700, 9,849,142 and 10,029,013 (to Foamix Pharmaceuticals Ltd.), claiming surfactant-free foamable compositions. The compositions of the above patents comprise, in addition to minocycline as part of a list of active agents, a large number of ingredients, belonging to a number of ingredient types, such as waxes, fatty acids, shea butter, short chain alcohols, polyols, polar solvents, polymers, hydrocarbon-based oils, mineral oils and petrolatum. Waxes, fatty acids and shea butter are important ingredients, present in most compositions of the above patents. Many of the above ingredients and ingredient types in the above patents are irritants or deleterious to the skin or to the active agent and should be avoided in a topical formulation.

For example, the compositions in U.S. Pat. No. 8,945,516 comprise instead of a surfactant an “oleaginous foamer complex”, which is a mixture of a fatty alcohol with various combinations of ingredients selected from waxes, fatty acids, shea butter, petrolatum and hydrocarbon-based oils.

The above patent states (column 3, rows 7-10) that “surface active agents can be advantageously eliminated and replaced by foam adjuvants and waxes in the context of hydrophobic solvent based-foams”.

U.S. Pat. No. 8,945,516 states in Example 4, column 63, lines 37-39 that “fatty alcohols alone are not sufficient as foaming agents in oleaginous formulations”.

This disclosure teaches away from using fatty alcohols alone as foaming agents, as the inventors describe in the present disclosure.

The development of a physically and chemically stable foamable minocycline composition proved to be a difficult task. Many of the compositions investigated did not comply with the requirements of a stable commercial product having long shelf-life, good quality foam and pharmaceutically acceptable minocycline assay stability on storage. Some of the compositions investigated exhibited poor physical stability (phase separation), some others led to poor quality foams (see Table 3).

### Impact of the Hydrophobic Oils (HO) to Fatty Alcohols (FA) Ratio on Foam Quality

A series of experiments (Examples 10-19, Table 3) was carried out with a view to determine the impact of the hydrophobic oils to fatty alcohols ratio on the foam quality.

All compositions of Examples 10-19 are essentially free of water, waxes, fatty acids, shea butter, short chain alcohols, polyols, polar solvents, polymers, hydrocarbon-based oils, mineral oils and petrolatum.

The compositions investigated comprised three hydrophobic oils (cyclomethicone, coconut oil and soybean oil) and three fatty alcohols (myristyl alcohol, behenyl alcohol and cetyl alcohol), in various percentages and ratios. In this series of experiments, the main variable selected was the concentration of the main fatty alcohol, cetyl alcohol.

The hydrophobic oils to fatty alcohols ratio was calculated by dividing the sum of the three hydrophobic oils w/w percentages to the sum of the three fatty alcohols percentages in the compositions. The compositions cover hydrophobic oils to fatty alcohols ratio range from about 3:1 to about 13.5:1 in the six different compositions of Examples 10-19 in Table 3.

The results in Table 3 show that the hydrophobic oils to fatty alcohols ratio has a dramatic effect on the appearance and quality of the foam obtained by adding propellant (AP-70) to each of the compositions, in a ratio of 10:100 as detailed in Example 6.

Surprisingly, the composition with hydrophobic oils to fatty alcohols ratio of about 4:1 to 8:1 exhibited satisfactory results of a stable foam, more preferably in a ratio of between 5:1 to 7.5:1 exhibited the most satisfactory results out the 10 experiments series. In another embodiment, the hydrophobic oils to fatty alcohols ratio is 4:1, 5:1, 5.1:1, 5.2:1, 5.3:1, 5.4:, 5.5:1, 5.6:1, 5.7:1, 5.8:1, 5.9:1, 6:1, 7:1, 7.1:1, 7.2:1, 7:3, 7.4:1, 7.5:1, 7.6:1, 7.7:1, 7.8:1, 7:9:1, 8:1, each represent a separate embodiment of this invention. In another embodiment, the hydrophobic oils to fatty alcohols ratio is between 4:1 to 7.9:1. Compositions with a ratio higher than 8:1 or equal to or lower than 3.9 exhibited unsatisfactory foam quality and appearance (see Table 3) on addition of propellant.

The compositions of Table 1, which were tested in accelerated stability studies and exhibited good foam appearance and quality have hydrophobic oils to fatty alcohols w/w ratio of from about 5:1 to about 7.5:1.

The present inventors have surprisingly discovered that a simple composition comprising in addition to the at least one active agent and hydrophobic oils, only one type of ingredient, fatty alcohols, is foamable, stable and effective. Fatty alcohols and hydrophobic oils are mild, not irritant or deleterious to the skin or the active ingredient yet the compositions are stable, foamable and effective, as proved by the minocycline compositions disclosed in Examples 1-8, 9 and Tables 1 and 2.

The stability of the compositions of this invention is better than the stability of comparable composition 244A detailed in U.S. Pat. No. 8,945,516 (vide infra).

The simple compositions of this invention lead also to an improved ease of manufacturing.

The “foamer complex” claimed in U.S. Pat. No. 8,945,516, which uses ingredients from the types of waxes, fatty acids, shea butter and polymers, is absent in the composition of this invention, which uses instead only fatty alcohols.

The fact that fatty alcohols alone, in absence of waxes, fatty acids, shea butter and polymers lead to stable, foamable and effective foamable compositions is novel and surprising.

Also novel and surprising is the finding that the ratio between the total w/w percentage of hydrophobic oils (HO) to the total w/w percentage of fatty alcohols (FA) in the compositions of this invention has such a dramatic effect on the foam quality of the composition, after addition of propellant. It was surprisingly found that a HO/FA ratio from about 4:1 to about 8:1 is optimal.

The composition of this invention may further comprise from about 0.1% to about 0.5% by weight of the composition of a thickener, like silicone oxide. The silicon oxide is typically Syloid™ or Aerosil™.

The composition of this invention may further comprise from about 0.1% to about 20% by weight of the composition of at least one fatty acid selected from hexadecanoic acid heptadecanoic acid, stearic acid, palmitic acid, arachidic acid, behenic acid, tetracosanoic acid, hexacosanoic acid, heptacosanoic acid, octacosanoic acid, triacontanoic acid, dotriacontanoic acid, tritriacontanoic acid, tetratriacontanoic acid, pentatriacontanoic acid, dodecanoic acid, myristic acid, myristoleic acid, lauric acid and mixtures thereof, and from 0.1% to about 5% by weight of the composition of a polymer.

In some embodiments, the polymer is selected from the group consisting of a polypropylene glycol, polyethylene glycol, ethylcellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

A typical polymer is a polyethylene glycol. Examples 5-8 use as polymer the polyethylene glycol PEG 3350.

The fatty acid used in the compositions disclosed in Examples 2, 3, 5-8 is commercial stearic acid, which is a mixture of stearic and palmitic acids. The compositions disclosed in Examples 5-8 comprise, in addition to stearic acid, also the polyethylene glycol polymer PEG 3350.

The composition of this invention may further comprise at least one propellant, wherein the ratio of composition to propellant is from about 100:3 to about 100:30. preferably 100:10, and wherein upon dispensing, the foamable composition forms a breakable foam that breaks easily upon application of shear force.

The compositions of Examples 1-8 comprise propellant AP-70 (55% propane, 18% isobutane, 27% n-butane) in a /composition/propellant ratio of 100:10.

The at least one hydrophobic oil in the compositions of this invention is selected from the group consisting of a therapeutic oil, an alexandria laurel tree oil, an almond oil, an essential oil, an unsaturated or polyunsaturated oil, an apricot stone oil, an avocado oil, a barley oil, a basil oil, a borage seed oil, a calendula oil, a camphor oil, a canelle nut tree oil, a canola oil, a cardamom oil, a carrot oil, a castor oil, a citronella oil, a clary sage oil, a clove oil, a coconut oil, a cod-liver oil, a corn oil, a cotton oil, a cottonseed oil, a cypress oil, a cyclomethicone oil, an epoxy-modified silicone oil, an ester oil, an evening primrose oil, a fatty acid-modified silicone oil, a flaxseed oil, a fluoro group-modified silicone oil, a frankincense oil, a ginger oil, a grape seed oil, a grapefruit oil, a groundnut oil, a hazelnut oil, a hempseed oil, a herring oil, a hyssop oil, a jasmine oil, a jojoba oil, a lavender oil, a lemon oil, a lucerne oil, a maize germ oil, a maleated soybean oil, a mandarin oil, a manuka oil, a marjoram oil, a marrow oil, a MCI oil, a millet oil, a myrrh oil, a neroli oil, a nutmeg oil, oils from animal origin, oils of plant origin, an olive oil, a palm oil, a passionflower oil, a peanut oil, a petitgrain oil, a polyether group-modified silicone oil, a poppy oil, a rapeseed oil, a rosehip oil, a rye oil, a safflower oil, a sage oil, a salmon oil, a sesame oil, a silicone oil, a soybean oil, a soybean oil, a sunflower oil, a sweet almond oil, a sysymbrium oil, a syzigium aromaticum oil, a tangerine oil, a tea tree oil, unsaturated or polyunsaturated oils, a vanilla oil, a verbena oil, a walnut oil, a wheat germ oil, and mixtures of any two or more thereof.

The exemplified compositions (Examples 1-8) comprise hydrophobic oils as a mixture of coconut oil, soybean oil and cyclomethicone in various percentages as indicated in Table 1.

The fatty acid and polymer free minocycline compositions of this invention comprise from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 5% w/w to about 15% w/w cetyl alcohol, from about 0.5% w/w to about 2% w/w behenyl alcohol and optionally from about 0.1% w/w to about 0.5% w/w Syloid™ (see Examples 2 and 3).

The fatty acid containing minocycline compositions of this invention comprise from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 10% w/w to about 20% w/w cetyl alcohol, from about 0.5% w/w to about 2% w/w behenyl alcohol, from about 5% w/w to about 8% w/w stearic/palmitic acid mixture, and optionally from about 0.1% to about 10% by weight of polyethylene glycol 3350 and from about 0.1% w/w to about 0.5% w/w Syloid™ (see Examples 2, 3, 5-8).

The stability of the compositions of this invention was determined in an accelerated stability study for 3 months at 40° C./75% RH and measured by an HPLC method as detailed in Example 9 and Table 2.

All formulations tested for stability (see Table 2) were found chemically stable according to their HPLC assay results compared to the initial measurement (t=0).

The at least one hydrophobic oil and the at least one fatty alcohol are present in the compositions of this invention in sufficient amounts and ratios to ensure the stability of the minocycline in the composition for at least about 3 months under accelerated stability conditions at 40° C./75% RH.

Examples 1-8 disclosed in Tables 1 and 2 and Examples 10-19 in Table 3 encompass a range of amounts and ratios of the at least one hydrophobic oil and the at least one fatty alcohol.

The stability and foam appearance of the compositions of this invention depend on the amounts and ratios of the at least one hydrophobic oil and the at least one fatty alcohol in the specific compositions.

Thus, for example, the compositions of Examples 3 and 4 (about 1.5% minocycline at t=0, Tables 1 and 2) exhibit exceptional chemical stability (assay loss vs t=0 of 0.69% and 1.4% respectively) under 3 months accelerated stability conditions at 40° C./75% RH.

The above chemical stability results of the compositions of Examples 3 and 4 of this invention (about 1.5% minocycline) are much better than those of a similar minocycline composition of U.S. Pat. No. 8,945,516 (Table 13b(i), composition 244B, 1.11% minocycline), comprising a foamer complex including i.a. a fatty acid and a wax. The accelerated stability study of the 244B composition showed a minocycline assay loss of 5% (93.8% vs. 98.7% of label claim at t=0) after 3 months under accelerated stability conditions at 40° C. and a minocycline assay loss of 8.5% (90.3% vs. 98.7% of label claim at t=0) after 6 months under accelerated stability conditions at 40° C.

None of the compositions of Examples 1-8 contain waxes or shea butter. Some of the compositions of Examples 1-8 contain a fatty acid and polymer, and their effect on the stability was studied. Examples 1 and 4 are essentially free of waxes, fatty acids, shea butter, short chain alcohols, polyols, polar solvents, polymers, hydrocarbon-based oils, mineral oils and petrolatum.

Noteworthy, while the addition of a fatty acid (in Example 3) did not negatively impact the minocycline chemical stability, the composition free of foamer complex, fatty acids, waxes, shea butter and polymers (in Examples 1 and 4) showed excellent stability and foam appearance (Table 2). This proves that composition comprising fatty alcohols as the sole type of foaming agent exhibit excellent chemical stability.

The compositions of Examples 1 and 2 (about 4% minocycline at t=0, Tables 1 and 2) exhibit very good stability (minocycline assay loss of 1.5-2.0% vs. t=0) under 3 months accelerated stability conditions at 40° C./75% RH.

The compositions of Examples 5 and 6 were tested for the time being only for 2 weeks under accelerated stability conditions at 40° C./75% RH and showed very good stability (less than 0.5% assay loss vs. t=0).

The compositions of Examples 7 and 8 (formulations 12 and 13), containing a higher concentration of polymer, were physically unstable (two-phase separation).

4-Epiminocycline (EMC), an impurity of the minocycline raw material. is the main impurity in the compositions, appearing in the HPLC at relative retention time RRT 1.04.

Two other minor unidentified impurities, also present in the minocycline raw material, appear at RRTs 0.79 and 0.96.

Impurity content was measured by HPLC peak areas at each impurity HPLC relative retention time (RRT).

Effect of the polymer in the compositions: the polymer modifies the texture of the foam, increasing the viscosity.

In some embodiments, there are provided stable, foamable and effective minocycline compositions essentially free of a “foamer complex”, waxes, fatty acids, shea butter, short chain alcohols, polyols, polar solvents, polymers, hydrocarbon-based oils, mineral oils and petrolatum, which exhibit good or excellent stability and good foam appearance after at least 3 months in an accelerated stability test at 40° C. at 75% relative humidity (RH). Minocycline in the composition of this invention, does not decompose more than 3% for at least about 3 months under accelerated stability conditions at 40° C./75% RH.

The stability of the compositions of this invention is better than the stability of previously published comparable compositions.

In some embodiments, there is provided a stable composition comprising:

from about 1% to about 5% by weight of at least one active agent:
from about 60% to about 90% by weight of the composition of at least one hydrophobic oil, and
from about 5% to about 25% by weight of the composition of at least one fatty alcohol
wherein the at least one hydrophobic oil and the at least one fatty alcohol comprise in total from about 65% to from about 99% by weight of the composition; and
wherein the at least one hydrophobic oil and the at least one fatty alcohol are sufficient to ensure the pharmaceutically acceptable assay stability of the at least one active agent in the composition under about 3 months accelerated stability conditions at 40° C./75% RH and about 2 years stability at room temperature. In another embodiment, the composition is a foamable composition and further comprises a propellant.

In some embodiments, the compositions of this invention are essentially free of water, waxes, fatty acids, shea butter, short chain alcohols, polyols, polar solvents, polymers, hydrocarbon-based oils, mineral oils and petrolatum.

In some embodiments, the at least one active agent is selected from the group consisting of adapalene, adipic acid, an acaricide, an active herbal extract, an age spot and keratose removing agent, an alpha hydroxy acid, an analgesic agent, an androgen, an anesthetic, an anti-wrinkle agent, an antiacne agent, an antiaging agent, an antiallergic agent, an antiandrogen agent, an antiapoptotic agent, an antibacterial agent, an antibiotic, an anti-burn agent, an anticancer agent, an anti-dandruff agent, an antidepressant, an anti-dermatitis agent, an anti-edemic anent, an antifungal agent, an antihelminth agent, an antihistamine, an anti-hyperkeratosis agent, an anti-infective agent, an anti-inflammatory agent, an anti-irritant, an antilipemic agent, an antimicrobial agent, an antimycotic agent, an antioxidant, an antiparasitic agent, an anti-photoaging agent, an anti-photodamaging agent, an antiproliferative agent, an antipruritic agent, an anti-psoriatic agent, an anti-rosacea agent, an anti-seborrheic agent, an antiseptic agent, an anti-swelling agent, an antiviral agent, an anti-wart agent, an anti-wrinkle agent, an anti-yeast agent, azelaic acid, benzoyl peroxide, a beta-hydroxy acid, calcitriol, a cardiovascular agent, a chemotherapeutic agent, a corticosteroid, a dicarboxylic acid, a dihydrotestosterone inhibitor, a disinfectant, doxycycline, an estrogen, a fungicide, fumaric acid, glycolic acid, a hair growth regulator, a haptene, a herbal extract (comprising an active substance), a hormone, a hydroxy acid, an immunogenic substance, an immunomodulator, an immuno-regulating agent, an immunostimulant, an immunosuppressant, an immuno-suppressive agent, an insect repellent, an insecticide, iron oxide, ivermectin, a keratolytic agent, lactic acid, a lactam, lidocaine, a local anesthetic agent, a minocycline, a mitocide, mometasone furoate, a neuropeptide, a non-steroidal anti-inflammatory agent, an organo-metallic compound, an oxidizing agent, and organo-boron compound, a pediculicide, a peptide, a pesticide, a photodynamic therapy agent, a progesterone, a prostaglandin, a protein, a radical scavenger, a retinoid, a sedative agent, a scabicide, sebacic acid, a sedative, a sedative agent, a self-tanning agent, silver, a silver compound, a skin protective agent, a skin whitening agent, a steroid, a steroidal anti-inflammatory agent, tretinoin, tazarotene, a testosterone inhibitor, a tetracycline antibiotic, a vasoactive agent, a vasoconstrictor, a vasodilator and mixtures thereof.

According to some embodiments, there is provided a stable composition comprising: from about 1% to about 5% by weight of a minocycline from about 60% to about 90% by weight of the composition of at least one hydrophobic oil, and from about 5% to about 25% by weight of the composition of at least one fatty alcohol, wherein the at least one hydrophobic oil and the at least one fatty alcohol comprise in total from about 65% to about 99% by weight of the composition;

wherein the at least one hydrophobic oil and the at least one fatty alcohol are in a weight ratio of from about 4:1 to about 8:1;
wherein the composition is essentially free of water, waxes, fatty acids, shea butter, short chain alcohols, polyols, polar solvents, polymers, hydrocarbon-based oils, mineral oil and petrolatum; and
wherein minocycline does not decompose more than 3% for at least about 3 months under accelerated stability conditions at 40° C./75% RH. In another embodiment, the composition is a foamable composition and further comprises a propellant.

The minocycline in the above composition is selected from minocycline, minocycline hydrochloride, a hydrate, a solvate and mixtures thereof.

According to some embodiments, the composition further comprises at least one propellant, wherein the ratio of composition to propellant is from about 100:3 to about 100:30, preferably 100:10, and wherein upon dispensing, the foamable composition forms a breakable foam that breaks easily upon application of shear force. The propellant is selected from n-butane, isobutane, propane, n-pentane and mixtures thereof.

In certain embodiments, the composition of this invention comprises from about 1% to about 5% by weight of the composition of a minocycline or mixtures thereof, from about 60% to about 90% by weight of the composition of at least one hydrophobic oil, from about 5% to about 25% by weight of the composition of at least one fatty alcohol, further comprising from about 0.1% to about 0.5% by weight of the composition of silicone oxide and a propellant in a ratio of composition to propellant from about 100:3 to about 100:30, preferably 100:10.

The composition of this invention may further comprise from about 0.1% to about 0.5% by weight of the composition of silicone oxide.

In some embodiments, the composition of this invention may further comprise from about 0.1% to about 20% by weight of the composition of at least one fatty acid selected from hexadecanoic acid heptadecanoic acid, stearic acid, palmitic acid, arachidic acid, behenic acid, tetracosanoic acid, hexacosanoic acid, heptacosanoic acid, octacosanoic acid, triacontanoic acid, dotriacontanoic acid, tritriacontanoic acid, tetratriacontanoic acid, pentatriacontanoic acid, dodecanoic acid, myristic acid, myristoleic acid, lauric acid and mixtures thereof. and from 0.1% to about 10% by weight of the composition of a polymer.

In some other embodiments, the at least one fatty acid is selected from stearic acid, palmitic acid and mixtures thereof.

According to some embodiments, the polymer in the above composition is selected from the group consisting of a polypropylene glycol, polyethylene glycol, ethyl cellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

In some embodiments, the at least one hydrophobic oil in the composition of this invention is selected from the group consisting of a therapeutic oil, an alexandria laurel tree oil, an almond oil, an essential oil, an unsaturated or polyunsaturated oil, an apricot stone oil, an avocado oil, a barley oil, a basil oil, a borage seed oil, a calendula oil, a camphor oil, a canelle nut tree oil, a canola oil, a cardamom oil, a carrot oil, a castor oil, a citronella oil, a clary sage oil, a clove oil, a coconut oil, a cod-liver oil, a corn oil, a cotton oil, a cottonseed oil, a cyclomethicone oil, a cypress oil, an epoxy-modified silicone oil, an ester oil, an evening primrose oil, a fatty acid-modified silicone oil, a flaxseed oil, a fluoro group-modified silicone oil, a frankincense oil, a ginger oil, a grape seed oil, a grapefruit oil, a groundnut oil, a hazelnut oil, a hempseed oil, a herring oil, a hyssop oil, a jasmine oil, a jojoba oil, a lavender oil, a lemon oil, a lucerne oil, a maize germ oil, a maleated soybean oil, a mandarin oil, a manuka oil, a marjoram oil, a marrow oil, a MCT oil, a millet oil, a myrrh oil, a neroli oil, a nutmeg oil, oils from animal origin, oils of plant origin, an olive oil, a palm oil, a passionflower oil, a peanut oil, a petitgrain oil, a polyether group-modified silicone oil, a poppy oil, a rapeseed oil, a rosehip oil, a rye oil, a safflower oil, a sage oil, a salmon oil, a sesame oil, a silicone oil, a soya oil, a soybean oil, a sunflower oil, a sweet almond oil, a sysymbrium oil, a syzigium aromaticum oil, a tangerine oil, a tea tree oil, unsaturated or polyunsaturated oils, a vanilla oil, a verbena oil, a walnut oil, a wheat germ oil, and mixtures of any two or more thereof.

According to some embodiments, the at least one hydrophobic oil in the composition of this invention is selected from coconut oil, soybean oil, a cyclomethicone and mixtures thereof.

In some embodiments, there is provided a composition comprising at least one hydrophobic oil consisting of from about 20% to about 30% coconut oil, from about 45% to about 55% soybean oil and from about 4% to about 6% of a cyclomethicone.

In some other embodiments, the at least one fatty alcohol in the composition of this invention is selected from myristyl alcohol, cetyl alcohol, behenyl alcohol and mixtures thereof.

According to some embodiments, there is provided a composition comprising at least one fatty alcohol consisting of from about 2% to about 3% myristyl alcohol, from about 5% to about 15% cetyl alcohol and from about 0.5% to about 2% behenyl alcohol.

According to other embodiments, the composition of this invention may further comprise at least one additional ingredient selected from about 5% w/w to about 8% w/w stearic acid, from about 0.1% to about 5% by weight of polyethylene glycol 3350, from about 0.1% w/w to about 0.5% w/w silicone dioxide or two or more thereof.

In some embodiments, there is provided a composition of this invention, wherein the composition comprises from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 5% w/w to about 15% w/w cetyl alcohol, from about 0.5% w/w to about 2% w/w behenyl alcohol and from about 0.1% w/w to about 0.5% w/w silicone dioxide.

In some other embodiments, there is provided a composition of this invention, wherein the composition comprises from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 10% w/w to about 20% w/w cetyl alcohol, from about 0.5% w/w to about 2% w/w behenyl alcohol, from about 5% w/w to about 8% w/w stearic acid, from about 0.1% to about 5% by weight polyethylene glycol 3350 and from about 0.1% w/w to about 0.5% w/w silicone dioxide.

According to some embodiments, there is provided a method of treatment of a skin disorder selected from acne, rosacea and impetigo, by administration to a subject in need thereof a therapeutically effective amount of the composition of this invention.

According to some embodiments, there is provided a method of treatment of a skin disorder selected from acne, rosacea and impetigo, by administration to a subject in need thereof a therapeutically effective amount of a stable composition comprising:

from about 1% to about 5% by weight of a minocycline; from about 60% to about 90% by weight of the composition of at least one hydrophobic oil; and
from about 5% to about 25% by weight of the composition of at least one fatty alcohol;
wherein the at least one hydrophobic oil and the at least one fatty alcohol comprise in total from about 65% to about 99% by weight of the composition;
wherein the at least one hydrophobic oil and the at least one fatty alcohol are in a weight ratio of from about 4:1 to about 8:1; and
wherein the composition is essentially free of water, waxes, fatty acids, shea butter, short chain alcohols, polyols, polar solvents, polymers, hydrocarbon-based oils, mineral oils and petrolatum. In another embodiment, the composition is a foamable composition and further comprises a propellant; and wherein minocycline does not decompose more than 3% for at least about 3 months under accelerated stability conditions at 40° C./75% RH. The minocycline in the above composition is selected from minocycline, minocycline hydrochloride, a hydrate, a solvate and mixtures thereof.

According to some other embodiments, the above skin disorder is acne and the composition comprise from about 1% w/w to about 5% w/w minocycline or its salt, hydrate, solvate or mixtures thereof. In another embodiment, from about 1% w/w to about 4% w/w minocycline or its salt, hydrate, solvate or mixtures thereof. In another embodiment the composition comprises 1%, 1.5%, 2%, 3%, 4% or 5% minocycline or its salt, hydrate, solvate or mixtures thereof. In another embodiment, the salt is hydrochloride salt.

In some embodiments, the above skin disorder is rosacea and the composition comprise from about 1% w/w to about 3% w/w minocycline or its salt, hydrate, solvate or mixtures thereof. In another embodiment, from about 1% w/w to about 4% w/w minocycline or its salt, hydrate, solvate or mixtures thereof. In another embodiment the composition comprises 1%, 1.5%, 2%, 3%, 4% or 5% minocycline or its salt, hydrate, solvate or mixtures thereof. In another embodiment, the salt is hydrochloride salt.

In some other embodiments, the above skin disorder is impetigo and the composition comprise from about 1% w/w to about 5% w/w minocycline or its salt, hydrate, solvate or mixtures thereof. In another embodiment, from about 1% w/w to about 4% w/w minocycline or its salt, hydrate, solvate or mixtures thereof. In another embodiment the composition comprises 1%, 1.5%, 2%, 3%, 4% or 5% minocycline or its salt, hydrate, solvate or mixtures thereof. In another embodiment, the salt is hydrochloride salt.

In some embodiments, there is provided the above minocycline composition, further comprising a therapeutically effective amount of at least one additional active agent selected from the group consisting of an anti-acne agent, tretinoin, adapalene, tazarotene, benzoyl peroxide and mixtures thereof.

In some other embodiments, the compositions of this invention may optionally further comprise from about 0.1% to about 0.5% by weight of the composition of silicone oxide. The silicon oxide used is typically Syloid™ or Aerosil™.

According to some embodiments. the compositions of this invention may optionally further comprise from about 0.1% to about 20% by weight of the composition of at least one fatty acid selected from hexadecanoic acid heptadecanoic acid, stearic acid, palmitic acid, arachidic acid, behenic acid, tetracosanoic acid, hexacosanoic acid, heptacosanoic acid, octacosanoic acid, triacontanoic acid, dotriacontanoic acid, tritriacontanoic acid, tetratriacontanoic acid, pentatriacontanoic acid, dodecanoic acid, myristic acid, myristoleic acid, lauric acid and mixtures thereof. and from 0.1% to about 10% by weight of the composition of a polymer.

In some embodiments, the polymer is selected from the group consisting of a polypropylene glycol, polyethylene glycol, ethylcellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

In some embodiments, the at least one fatty acid in the composition is selected from stearic acid, palmitic acid and mixtures thereof.

Unless otherwise defined, all technical and scientific terms used herein have the same meaning as commonly understood by one of ordinary skill in the art to which the invention pertains. In case of conflict, the specification, including definitions, takes precedence.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

As used herein, the term “treating” or“treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs

As used herein, the term “fatty alcohol” refers to a fatty acid alcohol having a carbon chain length of 14 to 22 carbons, a straight chain fatty alcohol, a saturated fatty alcohol, an unsaturated fatty alcohol, a hydroxyl substituted fatty alcohol or a branched fatty alcohol.

As used herein, the term “light mineral oil” refers to a mineral oil of CAS No. 92062356, as approved by the FDA's IIG database for Approved Drug Products.

The terms “hydrocarbon oil” or “hydrocarbon-based oil” refer to an oily liquid formed wholly or partly of hydrocarbons. Examples of hydrocarbon oils include light or heavy mineral oil, liquid paraffin, an isoparaffin, a polyalphaolefin, a polyolefin, polyisobutylene, a synthetic isoalkane, isohexadecane and isododecane.

The term “wax” refers to a liquid wax, a solid wax, an animal wax, a vegetable wax, a mineral wax, a natural wax or a synthetic wax. Exemplary waxes are paraffin wax, beeswax, hydrogenated castor oil or mixtures thereof.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, a “foamable composition” is a composition which on addition of a propellant, forms a breakable foam that breaks upon application of shear force.

As used herein, “poor foam appearance” relates to a foam collapsing in less than 3 minutes and/or to a discolored foam. Minocycline discolored foams tend to be brownish.

As used herein, the term “essentially free” generally refers to a composition having less than about 2 percent by weight, more preferably 1 percent per weight, less than about 0.5 percent by weight or even less than 0.1 percent by weight of a certain ingredient, based on the total weight of the composition.

Pharmaceutical compositions used in implementing the teachings herein may be formulated using techniques with which one of average skill in the art is familiar in a conventional manner using one or more pharmaceutically-acceptable compositions comprising excipients and adjuvants, which facilitate processing of the active ingredients into a pharmaceutical composition and generally includes mixing an amount of the active ingredients with the other components. Suitable techniques are described in “Remington's Pharmaceutical Sciences,” Mack Publishing Co., Easton, Pa., latest edition, which is incorporated herein by reference. For example, pharmaceutical compositions useful in implementing the teachings herein may be manufactured by one or more processes that are well known in the art, e.g., mixing, blending, homogenizing, dissolving, granulating, emulsifying, encapsulating, entrapping and lyophilizing processes.

Pharmaceutical compositions suitable for implementing the teachings herein include compositions comprising active ingredients in an amount effective to achieve the intended purpose (a therapeutically effective amount). Determination of a therapeutically effective amount is well within the capability of those skilled in the art, for example, is initially estimated from animal models such as rats, mice, monkeys or pigs.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

### EXAMPLES

Exemplary embodiments of the teachings herein are discussed hereinbelow with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

### Materials

Cyclomethicone NF (Dow Corning) assay 98.1%
Coconut oil (refined) EP (Henry Lamotte Oils) 0.0% water
Soybean oil USP (Spectrum) 0.0% water
Myristyl alcohol USP/NF (Kolliwax® MA BTC) 0.2% water
Behenyl alcohol (Lanette® 22 BASF)
Cetyl alcohol EP ((Kolliwax® CA BTC) 0.1% water
Silicone dioxide NF (Syloid® 244 FP)
Stearic acid NF (Spectrum Chemical Mfg. Corp.) 50.05% w/w stearic acid,
assay stearic & palmitic acid 99.6%.
Polyethylene glycol 3350 USP (PEG 3350—Integrated Quality Program IQ), water 0.1%
Minocycline hydrochloride micronized USP (Hovione), 7.4% water, 911 mcg/mg w/w
Propellant AP-70 (27% n-butane, 18% isobutane, 55% propane) Synthethis Chimica)

### General Procedure for the Preparation of the Compositions of Examples 1-8

### Example 6

Cyclomethicone (24 g), coconut oil (123 g), soybean oil (254.5 g), myristyl alcohol (12.5 g), cetyl alcohol (35 g), behenyl alcohol (5.5 g), silicone dioxide (1.25 g, SiO₂) stearic acid (stearic/palmitic acid mixture, 33.75 g) and PEG 3350 (3 g) were weighed in a beaker.

The obtained mixture was transferred to a 70° C. water bath and mixed with a magnetic stirrer until the ingredients were fully dissolved. After the full dissolution, the bath temperature was reduced to 50° C.

Minocycline HCl (7.5 g) was weighed in a weighing dish and was added to the above solution of the other ingredients in the beaker at 50° C. while stirring at high shear (7000 rpm) during 5 minutes.

The contents of the beaker were transferred to an ice-cooled water bath and cooled to room-temperature under manual mixing. The composition of Example 6 (about 500 g) was obtained.

A 40 g portion of the above composition was filled into a 100 mL Nussbaum aerosol can, then a valve was crimped to the can. After crimping the valve, 4 g of propellant AP-70 (27% n-butane, 18% isobutane, 55% propane, Synthethis Chimica) was added to the can, Samples were analyzed at t=0 and after 2 weeks, 1 month and 3 months of storage in a stability oven at 40° C.

The compositions of Examples 1-8 were prepared by using the procedure detailed in Example 6 above, using the percentages detailed in Table 1.

### Examples 1-8

The compositions of Examples 1-8 are detailed in Table 1 below:

TABLE 1
Compositions of Examples 1-8

| | % w/w Ingredients in Examples' Compositions | | | | | | | |
|---|---|---|---|---|---|---|---|---|
| | Examples | | | | | | | |
| Ingredients | 1 | 2 | 3 | 4 | 5 | 6 | 7 | 8 |
| Cyclomethicone | 4.8 | 4.8 | 4.8 | 4.8 | 4.8 | 4.8 | 4.8 | 4.8 |
| Coconut oil | 25.35 | 23.6 | 24.6 | 26.35 | 23.6 | 24.6 | 24.1 | 22.60 |
| Soybean oil | 50.0 | 50.0 | 51.5 | 51.5 | 49.4 | 50.9 | 50.0 | 48.5 |
| Myristyl alcohol | 2.5 | 2.5 | 2.5 | 2.5 | 2.5 | 2.5 | 2.5 | 2.5 |
| Behenyl alcohol | 1.10 | 1.10 | 1.10 | 1.10 | 1.10 | 1.10 | 1.10 | 1.10 |
| Cetyl alcohol | 12.0 | 7.0 | 7.0 | 12.0 | 7.0 | 7.0 | 7.0 | 7.0 |
| SiO₂ | 0.25 | 0.25 | 0.25 | 0.25 | 0.25 | 0.25 | 0.25 | 0.25 |
| Stearic acid | — | 6.75 | 6.75 | — | 6.75 | 6.75 | 6.75 | 6.75 |
| PEG 3350 | — | — | — | — | 0.6 | 0.6 | 2.0 | 5.0 |
| Minocycline HCl | 4.0 | 4.0 | 1.5 | 1.5 | 4.0 | 1.5 | 1.5 | 1.5 |
| AP-70 to formula | 10:100 | 10:100 | 10:100 | 10:100 | 10:100 | 10:100 | 10:100 | 10:100 |
| ratio | | | | | | | | |

### Example 9

### Minocycline Stability Studies in the Compositions

### Scope

An analytical method for the determination of an assay and related compounds of Minocycline in a foamable formulation in the compositions of this invention.

### Methods

### Sample Preparation Procedure

Sample solutions were prepared in triplicate.

The propellant was removed and the propellant-free sample was extracted with isopropyl alcohol and HCl 0.1N for isolating minocycline and its related compounds, then the extract was analyzed by HPLC with UV detection at 266 nm, as compared with the external standard.

### Abbreviations

### IPA Isopropyl Alcohol

### MeOH Methanol

### ACN Acetonitrile

HCl Hydrochloric acid

### NMT Not More Than

### NLT Not Less Than

### WS Working Standard

NA Not available
RRT Relative retention time

### Equipment

Agilent 1100/1200 HPLC instrument with quaternary pump and PDA detector

### Standards and Reagents

| Standard/Reagent | Manufacturer | Cat. No. |
|---|---|---|
| Minocycline in-house standard | Hovione F.C.SA | |
| Water, HPLC grade | J. T. Baker | 4218 |
| HCl 10% | | |
| Acetonitrile, HPLC grade | Merck | 1000305 |
| Methanol, HPLC grade | Bio Lab | 13683504 |
| Isopropyl Alcohol, for analysis | Merck | 109634 |
| Potassium dihydrogen phosphate, | Merck | 104877 |
| for analysis | | |
| 1-Octanesulfonic acid, sodium salt, | Acros Organics | 384771000 |
| HPLC grade | | |
| ortho-Phosphoric acid 85% | Merck | 100573 |

### Analytical Procedure

Chromatographic conditions: A Poroshell 120, EC-C18, 2.7 μm, 4.6*100 mm column (Agilent Cat No. 695975-902) was used, with a gradient program based on three eluents: eluent A—phosphate buffer pH-2.3 with ion-pair reagent, eluent B—CAN, eluent C—methanol. Column temperature was 40° C., flow-rate was 0.7 ml/min and detection was done by UV at 266 nm.

### Minocycline Accelerated Stability Results of the Compositions of Examples 1-8

### Methodology

All samples, prepared as detailed above, were placed in 40° C./75% RH stability chambers, and were taken out for analysis at the reported time points.

All the formulations were prepared in open beakers (not under inert atmosphere)

### Summary of Accelerated Stability Results

All formulations tested for stability were found chemically stable according to assay results.

4-Epiminocycline (EMC), also present in the minocycline raw material. 4-Epiminocycline (EMC) is the main impurity in the compositions, appearing in the HPLC at RRT 1.04.

Two other unidentified impurities, also present in the minocycline raw material, appear at RRTs 0.79 and 0.96.

Examples 1 and 4 (containing only fatty alcohols, free of fatty acids) show good foam appearance after 3 months at 40° C./75% RH.

Effect of the polymer in the compositions: the polymer modifies the texture of the foam, increasing the viscosity.

The compositions of Examples 7 and 8, containing a higher concentration of polymer, were physically unstable (two-phase appearance).

TABLE 2A
Accelerated Stability Results of Examples 1-8

| | |
|---|---|
| Examples | Minocycline assay % |

| (Formulations) | t = 0 | t = 2 w | t = 1 m | t = 3 m |
|---|---|---|---|---|
| vs. reference | RT | 40 C. | 40 C. | 40 C. |
| Example 1 (F7) | 4.00 | 4.03 | 3.91 | 3.94 |
| Example 2 (F6) | 4.00 | 3.97 | 3.94 | 3.92 |
| Example 3 (F8) | 1.45 | 1.48 | 1.43 | 1.44 |
| Example 4 (F9) | 1.48 | 1.48 | 1.44 | 1.46 |
| Example 5 (F10) | 3.88 | 3.86 | NA | NA |
| Example 6 (F11) | 1.46 | 1.45 | NA | NA |

| | |
|---|---|
| Example 7 (F12) | Phase separation |
| Example 8 (F13) | Phase separation |

TABLE 2B
Stability Results of a Composition of Example 1

| | | Formulation Example 1 | | | | |
|---|---|---|---|---|---|---|
| Parameter | | T = 0 | 40° C. 2 w | 40° C. 1 m | 40° C. 3 m | 40° C. 6 m |
| Minocycline assay, % | | 4.00 | 4.03 | 3.91 | 3.94 | 3.94 |
| Minocycline | RRT 0.79 | 0.12 | 0.13 | 0.15 | 0.13 | 0.13 |
| related | RRT 0.96 | 0.31 | 0.33 | 0.32 | 0.5 | 0.5 |
| compounds, | RRT 1.04 | 1.48 | 1.65 | 1.61 | 1.21 | 0.87 |
| % from | 4- | | | | | |
| label | epiminocycline | | | | | |
| amount | | | | | | |
| | | Yellow, looks | Yellow, looks | Yellow, looks | Yellow, looks | Brown, |
| | | good, a bit | good, a bit | good, a bit | good, a bit | stable |
| | | flaffy, stable | flaffy, stable | flaffy, stable | flaffy, stable | foam |
| | | foam | foam | foam | foam | |

TABLE 2C
Stability Results of a Composition of Example 2

| | | Formulation Example 2 | | | | | |
|---|---|---|---|---|---|---|---|
| Parameter | | T = 0 | 40° C. 2 w | 40° C. 1 m | 40° C. 3 m | 40° C. 6.5 m | 25° C. 6.5 m |
| Minocycline assay, % | | 4.00 | 3.97 | 3.94 | 3.92 | 3.97 | 3.97 |
| Minocycline | RRT 0.79 | 0.12 | 0.12 | 0.14 | 0.13 | 0.14 | 0.14 |
| related | RRT 0.96 | 0.31 | 0.32 | 0.31 | 0.38 | 0.39 | 0.39 |
| compounds, | RRT 1.04 | 2.31 | 1.92 | 1.77 | 1.24 | 1.41 | 1.41 |
| % from label | 4- | | | | | | |
| amount | epiminocycline | | | | | | |
| | | Yellow, looks | Yellow, looks | Yellow, looks | Yellow, | Brown, | Yellowish, |
| | | good, a bit | good, stable | good, stable | smooth, | stable | looks good, |
| | | flaffy, stable | foam | foam | soft | foam | stable |
| | | foam | | | foam | | foam |

TABLE 2D
Stability Results of a Composition of Example 3

| | | Formulation Example 3 | | | | | |
|---|---|---|---|---|---|---|---|
| Parameter | | T = 0 | 40° C. 2 w | 40° C. 1 m | 40° C. 3 m | 40° C. 6.5 m | 25° C. 6.5 m |
| Minocycline assay, % | | 1.45 | 1.48 | 1.43 | 1.44 | 1.45 | 1.44 |
| Minocycline | RRT 0.79 | 0.11 | 0.14 | 0.15 | 0.16 | 0.16 | 0.14 |
| related | RRT 0.96 | 0.30 | 0.32 | 0.34 | 0.71 | 0.71 | 0.43 |
| compounds, | RRT 1.04 | 3.25 | 2.79 | 2.60 | 1.08 | 1.08 | 1.95 |
| % from | 4- | | | | | | |
| label | epiminocycline | | | | | | |
| amount | | | | | | | |
| | | Yellow, looks | Yellow, looks | Yellow, looks | Yellow, looks | Brown, | Yellowish, |
| | | good, a bit | good, stable | good, stable | good, soft | soft | looks good, |
| | | flaffy, stable | foam | foam | foam | foam | stable |
| | | foam | | | | | foam |

TABLE 2E
Stability Results of a Composition of Example 4

| | | Formulation Example 4 | | | | |
|---|---|---|---|---|---|---|
| Parameter | | T = 0 | 40° C. 2 w | 40° C. 1 m | 40° C. 3 m | 40° C. 6 m |
| Minocycline assay, % | | 1.48 | 1.48 | 1.44 | 1.46 | 1.45 |
| Minocycline | RRT 0.79 | 0.12 | 0.13 | 0.13 | 0.14 | 0.15 |
| related | RRT 0.96 | 0.31 | 0.31 | 0.33 | 0.40 | 0.57 |
| compounds, | RRT 1.04 | 2.01 | 2.46 | 2.38 | 1.78 | 1.19 |
| % from | 4- | | | | | |
| label | epiminocycline | | | | | |
| amount | | | | | | |
| | | Yellow, looks | Yellow, looks | Yellow, looks | Yellow, looks | Brown, |
| | | good, stable | good, stable | good, stable | good, stable | stable |
| | | foam | foam | foam | foam | foam |

TABLE 2F
Stability Results of a Composition of Example 5

| | | Formulation Example 5 | | | |
|---|---|---|---|---|---|
| Parameter | | T = 0 | 40° C. 1 w | 40° C. 1 M | 40° C. 3 M |
| Minocycline assay, % | | 3.88 | 3.86 | 3.91 | 3.97 |
| Minocycline | RRT 0.79 | 0.13 | 0.13 | 0.15 | 0.14 |
| related | RRT 0.96 | 0.29 | 0.32 | 0.36 | 0.43 |
| compounds, | RRT 1.04 | 4.43 | 3.93 | 2.03 | 1.28 |
| area % | 4- | | | | |
| | epiminocycline | | | | |
| | | Yellow, | Yellow, | Brownish, | Brown, stable |
| | | looks good, | looks good, | stable foam | foam |
| | | stable foam | stable foam | | |

TABLE 2G
Stability Results of a Composition of Example 6

| | | Formulation Example 6 | | | |
|---|---|---|---|---|---|
| Parameter | | T = 0 | 40° C. 1 w | 40° C. 1 M | 40° C. 3 M |
| Minocycline assay, % | | 1.46 | 1.45 | 1.46 | NT |
| Minocycline | RRT 0.79 | 0.13 | 0.14 | 0.16 | NT |
| related | RRT 0.96 | 0.29 | 0.30 | 0.35 | NT |
| compounds, | RRT 1.04 | 4.25 | 2.52 | 1.72 | NT |
| area % | 4- | | | | |
| | epiminocycline | | | | |
| | | Yellow, | Yellow, | Brownish, | Not |
| | | looks good, | looks good, | stable foam | stable |
| | | stable foam | stable foam | | |

### Examples 10-19

### Impact of the Hydrophobic Oils (HO) to Fatty Alcohols (FA) Ratio on Foam Quality

A series of experiments (Examples 10-19, Table 3) was carried out, with a view to determine the impact of the hydrophobic oils to fatty alcohols on the foam quality.

All the compositions in Examples 10-19 were prepared according to the procedure detailed in Example 6 and comprise 4% minocycline HCl. All compositions of Examples 10-19 are essentially free of water, waxes, fatty acids, shea butter, short chain alcohols, polyols, polar solvents, polymers, hydrocarbon-based oils, mineral oils and petrolatum.

The compositions investigated comprised three hydrophobic oils (cyclomethicone, coconut oil and soybean oil) and three fatty alcohols (myristyl alcohol, behenyl alcohol and cetyl alcohol), in various percentages and ratios. In this series of experiments, the main variable selected was the concentration of the main fatty alcohol, cetyl alcohol.

The hydrophobic oils/fatty alcohols ratio was calculated by dividing the sum of the three hydrophobic oils w/w percentages to the sum of the three fatty alcohols in the compositions, covering hydrophobic oils to fatty alcohols ratio range from about 3:1 to about 13.5:1 in the six different compositions 10-19 in Table 3.

The results in Table 3 show that the hydrophobic oils to fatty alcohols ratio has a dramatic effect on the appearance and quality of the foam obtained by adding propellant (AP-70) to each of the compositions, in a ratio of 10:100.

The composition with hydrophobic oils to fatty alcohols ratio of about 5:1 to 7.5:1 exhibited the most satisfactory results out the 10 experiments series.

TABLE 3
Impact of the Hydrophobic Oils to Fatty Alcohols Ratio on Foam Quality

| | Examples | | | | | | | | | |
|---|---|---|---|---|---|---|---|---|---|---|
| | 10 | 11 | 12 | 13 | 14 | 15 | 16 | 17 | 18 | 19 |
| Ingredients | Percentages below are indicated in w/w | | | | | | | | | |
| Cyclomethicone | 4.8 | 4.8 | 4.8 | 4.8 | 4.8 | 4.8 | 4.8 | 4.8 | 4.8 | 4.8 |
| Coconut oil | 21.35 | 22.85 | 23.4 | 25.35 | 27.85 | 29.85 | 27.35 | 26.35 | 24.35 | 23.85 |
| Soybean oil | 46.0 | 47.5 | 48.05 | 50 | 52.50 | 54.50 | 52.0 | 51.0 | 49.0 | 48.5 |
| Myristyl alcohol | 2.5 | 2.5 | 2.5 | 2.5 | 2.5 | 2.5 | 2.5 | 2.5 | 2.5 | 2.5 |
| Behenyl alcohol | 1.1 | 1.1 | 5 | 1.1 | 1.1 | 1.1 | 1.1 | 1.1 | 1.1 | 1.1 |
| Cetyl alcohol | 20 | 17 | 12 | 12 | 7 | 3 | 8 | 10 | 14 | 15 |
| Syloid ® (SiO2) | 0.25 | 0.25 | 0.25 | 0.25 | 0.25 | 0.25 | 0.25 | 0.25 | 0.25 | 0.25 |
| Minocycline HCl | 4 | 4 | 4 | 4 | 4 | 4 | 4 | 4 | 4 | 4 |
| Foam quality and | Viscous, | Viscous, | Viscous, | Viscous, | Foam | Liquid | Mixes good, | Mixes good. | Viscous, | Viscous, |
| appearance | dry, | dry, hard | dry, | stable, | collapsed | | stable | stable | dry, stable, | dry, stable, |
| | N.S. | to mix | N.S | good | | | | | hard to mix | hard to mix |
| HO/FA ratio | 3.1 | 3.7 | 3.9 | 5.1 | 8.0 | 13.5 | 7.3 | 6.0 | 4.4 | 4.1 |

Legend: N.S.—non-shakable

1. A stable minocycline composition comprising:
from about 1% to about 5% by weight of minocycline;
from about 60% to about 90% by weight of the composition of at least one hydrophobic oil, and
from about 5% to about 25% by weight of the composition of at least one fatty alcohol,
wherein the at least one hydrophobic oil and the at least one fatty alcohol comprise in total from about 65% to about 99% by weight of the composition,
wherein the at least one hydrophobic oil and the at least one fatty alcohol are in a weight ratio of from about 4:1 to about 8:1; and
wherein the composition is essentially free of water, waxes, fatty acids, shea butter, short chain alcohols, polyols, polar solvents, polymers, hydrocarbon-based oils, mineral oils and petrolatum.

2. The composition of claim 1, wherein the minocycline is selected from minocycline, minocycline hydrochloride, a hydrate, a solvate and mixtures thereof.

3. The composition of claim 1, wherein the composition is a foamable composition, and the composition further comprises at least one propellant,
wherein the ratio of composition to propellant is from about 100:3 to about 100:30, preferably 100:10, and wherein upon dispensing, the foamable composition forms a breakable foam that breaks easily upon application of shear force.

4. The composition of claim 3, wherein said minocycline does not decompose more than 3% for at least about 3 months under accelerated stability conditions at 40° C./75% RH.

5. The composition of claim 3, wherein the propellant is selected from n-butane, isobutane, propane, n-pentane and mixtures thereof.

6. The composition of claim 3, comprising from about 1% to about 5% by weight of the composition of a minocycline, from about 60% to about 90% by weight of the composition of at least one hydrophobic oil, from about 5% to about 25% by weight of the composition of at least one fatty alcohol, further comprising from about 0.1% to about 0.5% by weight of the composition of silicone oxide and a propellant in a ratio of composition to propellant from about 100:3 to about 100:30, preferably 100:10.

7. The composition of claim 1, further comprising from about 0.1% to about 20% by weight of the composition of at least one fatty acid selected from hexadecanoic acid heptadecanoic acid, stearic acid, palmitic acid, arachidic acid, behenic acid, tetracosanoic acid, hexacosanoic acid, heptacosanoic acid, octacosanoic acid, triacontanoic acid, dotriacontanoic acid, tritriacontanoic acid, tetratriacontanoic acid, pentatriacontanoic acid, dodecanoic acid, myristic acid, myristoleic acid, lauric acid and mixtures thereof, and from 0.1% to about 10% by weight of the composition of a polymer.

8. The composition of claim 7, wherein the at least one fatty acid is selected from stearic acid, palmitic acid and mixtures thereof.

9. The composition of claim 7, wherein the polymer is selected from the group consisting of a polypropylene glycol, polyethylene glycol, ethyl cellulose, alkylated guar gum, trimethylsiloxysilicate, alkyl-modified silicone, polyamide-modified silicone homopolymers and copolymers of alkyl methacrylates, alkyl acrylates and alkyl styrenes, polyisobutene, polybutyl methacrylate and polycyclohexylstyrene.

10. The composition of claim 1, wherein the at least one hydrophobic oil is selected from the group consisting of a therapeutic oil, an alexandria laurel tree oil, an almond oil, an essential oil, an unsaturated or polyunsaturated oil, an apricot stone oil, an avocado oil, a barley oil, a basil oil, a borage seed oil, a calendula oil, a camphor oil, a canelle nut tree oil, a canola oil, a cardamom oil, a carrot oil, a castor oil, a citronella oil, a clary sage oil, a clove oil, a coconut oil, a cod-liver oil, a corn oil, a cotton oil, a cottonseed oil, a cyclomethicone oil, a cypress oil, an epoxy-modified silicone oil, an ester oil, an evening primrose oil, a fatty acid-modified silicone oil, a flaxseed oil, a fluoro group-modified silicone oil, a frankincense oil, a ginger oil, a grape seed oil, a grapefruit oil, a groundnut oil, a hazelnut oil, a hempseed oil, a herring oil, a hyssop oil, a jasmine oil, a jojoba oil, a lavender oil, a lemon oil, a lucerne oil, a maize germ oil, a maleated soybean oil, a mandarin oil, a manuka oil, a marjoram oil, a marrow oil, a MCT oil, a millet oil, a myrrh oil, a neroli oil, a nutmeg oil, oils from animal origin, oils of plant origin, an olive oil, a palm oil, a passionflower oil, a peanut oil, a petitgrain oil, a polyether group-modified silicone oil, a poppy oil, a rapeseed oil, a rosehip oil, a rye oil, a safflower oil, a sage oil, a salmon oil, a sesame oil, a silicone oil, a soya oil, a soybean oil, a sunflower oil, a sweet almond oil, a sysymbrium oil, a syzigium aromaticum oil, a tangerine oil, a tea tree oil, unsaturated or polyunsaturated oils, a vanilla oil, a verbena oil, a walnut oil, a wheat germ oil, and mixtures of any two or more thereof.

11. The composition of claim 10, wherein the at least one hydrophobic oil is selected from coconut oil, soybean oil, a cyclomethicone and mixtures thereof.

12. The composition of claim 11, wherein the composition comprises at least one hydrophobic oil consisting of from about 20% to about 30% coconut oil, from about 45% to about 55% soybean oil and from about 4% to about 6% of a cyclomethicone.

13. The composition of claim 6, wherein the at least one fatty alcohol is selected from myristyl alcohol, cetyl alcohol, behenyl alcohol and mixtures thereof.

14. The composition of claim 13, wherein the composition comprises at least one fatty alcohol consisting of from about 2% to about 3% myristyl alcohol, from about 5% to about 15% cetyl alcohol and from about 0.5% to about 2% behenyl alcohol.

15. The composition of claim 12, further comprising at least one additional ingredient selected from about 5% w/w to about 8% w/w stearic acid, from about 0.1% to about 5% by weight of polyethylene glycol 3350 and from about 0.1% w/w to about 0.5% w/w silicone dioxide or two or more thereof.

16. The composition of claim 6, wherein the composition comprises from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 5% w/w to about 15% w/w cetyl alcohol, from about 0.5% w/w to about 2% w/w behenyl alcohol and from about 0.1% w/w to about 0.5% w/w silicone dioxide.

17. The composition of claim 6, wherein the composition comprises from about 1% w/w to about 5% w/w minocycline hydrochloride, from about 4% w/w to about 6% w/w cyclomethicone, from about 20% w/w to about 30% w/w coconut oil, from about 45% w/w to about 55% w/w soybean oil, from about 2% w/w to about 3% w/w myristyl alcohol, from about 10% w/w to about 20% w/w cetyl alcohol, from about 0.5% w/w to about 2% w/w behenyl alcohol, from about 5% w/w to about 8% w/w stearic acid, from about 0.1% to about 5% by weight polyethylene glycol 3350 and from about 0.1% w/w to about 0.5% w/w silicone dioxide.

18. A method of treatment of a skin disorder selected from acne, rosacea and impetigo, by administration to a subject in need thereof a therapeutically effective amount of the composition of claim 1.

19. The method of claim 18, wherein the composition comprises, from about 1% w/w to about 5% w/w of minocycline hydrochloride.

20. The method of claim 18, wherein the skin disorder is acne and the composition comprise from about 1% w/w to about 4% w/w minocycline.

21. The method of claim 18, wherein the skin disorder is rosacea and the composition comprise from about 1% w/w to about 3% w/w minocycline.

22. The method of claim 18, wherein the skin disorder is impetigo and the composition comprise from about 1% w/w to about 4% w/w minocycline.

23. The composition of claim 2, wherein the composition is a foamable composition, and the composition further comprises at least one propellant, wherein the ratio of composition to propellant is from about 100:3 to about 100:30, preferably 100:10, and wherein upon dispensing, the foamable composition forms a breakable foam that breaks easily upon application of shear force.

24. The composition of claim 4, wherein the propellant is selected from n-butane, isobutane, propane, n-pentane and mixtures thereof

25. The composition of claim 13, further comprising at least one additional ingredient selected from about 5% w/w to about 8% w/w stearic acid, from about 0.1% to about 5% by weight of polyethylene glycol 3350 and from about 0.1% w/w to about 0.5% w/w silicone dioxide or two or more thereof.
